(19) United States
(12) Patent Application Publication
(10) Pub. No.: US 2002/0166556 A1
(43) Pub. Date: Nov. 14, 2002

(54) Nose airflow enhancer and filter for airborne solids and pathogens

(76) Inventor: Joseph Jacob, Wooster, OH (US)

Correspondence Address: Warren N. Low, Esq., Low and Low, P.O. Box 2184, Arlington, VA, 22202, US

(21) Appl. No.: 09/851,343

(22) Filed: May 9, 2001

(51) Int. Cl. A62B 7/10; A62B 23/02; A61G 10/00; A61M 16/00

(52) U.S. Cl. 128/206.11; 128/205.27; 128/205.29

(57) ABSTRACT

A nose airflow enhancer and filter for airborne solids and pathogens utilizing a flexible plastic spring material having an attachment system to connect the spring material to the nose in a U-shaped configuration, with the plastic material underlying the nostrils, the spring material having attached thereto a length of filter material which underlies and is in substantial contact with the nose, thereby permitting the filter material to remove airborne solids and pathogens during breathing of the user while airflow through the nose is enhanced.

### BACKGROUND OF THE INVENTION

There has been extensive development of facemask filter assemblies in recent years to reduce inhalation of dust, contaminants, and various allergens. Such mask-like filters are uncomfortable, unduly warm, and are not always effective to reduce pathogens or contaminants, as diverse bacteria and dust mites. Further the same are often relatively large and ungainly for the user, and often somewhat difficult to don and remove.

### BRIEF DESCRIPTION OF THE INVENTION

The present invention makes great strides in improving filtration for the user in providing a disposable filter assembly which is in immediate proximity to the nostrils in use, utilizes available filter material which is capable of removing exceedingly small airborne particulates and allergenic substances. Further, the filter assembly is lightweight, is readily and quickly placed in protective position about the nostrils, and also enhances full breathing airflow by maintaining a nostril-opening force while in use.

To this end, the filter assembly includes a relatively small and narrow flexible plastic spine to which is affixed a shorter length of filter material. The plastic spine further includes a pair of pads at its ends which are provided with adhesive to emplace the filter assembly on the nose with the filter material extending under and around the nostrils as the user breathes. Simultaneously, the adhesive pads under the spring force of the spine tend to urge the nostrils to a fully open position. The filter includes a free or unattached end, which assists in accommodating noses and nostrils of varying sizes and configurations.

### BRIEF DESCRIPTION OF THE DRAWINGS

The invention will be better understood in connection with the accompanying drawings, in which:

FIG. 1 is a perspective view showing the nose filter in position on a user's nose to remove airborne solids and pathogens as the user breathes;

FIG. 2 is somewhat similar to FIG. 1, but looking downwardly toward the nose with the filter in place;

FIG. 3 is a side elevation of the filter before use, showing the general U-shape thereof when flexed for use; and,

FIG. 4 is a plan view looking upwardly with respect to FIG. 3 showing attachment of the filter material to the U-shaped spring material.

### DESCRIPTION OF THE PREFERRED EMBODIMENT

Referring to the drawings, the overall filter system is seen at 10, and includes a length of spring material 12, which is preferably of flexible plastic, such as a polyolefin, as polypropylene, for example. While the spring material 12 may take a variety of forms, in the preferred embodiment the same comprises an elongated narrow strip or spine 14 having a pair of transverse extensions 16 near the respective ends of the strip portion 14. The strip portion is of sufficient size and thickness to impart the necessary spring forces on the assembly in use. In one illustrative embodiment, the spine 14 is on the order of 2.20″ in length and 0.130″ in width, while the extensions 16 are 0.20″ in width and about 1.0″ in total height.

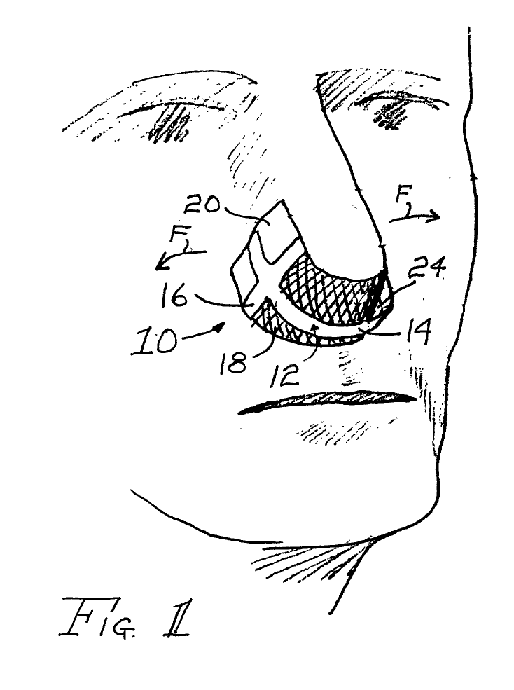

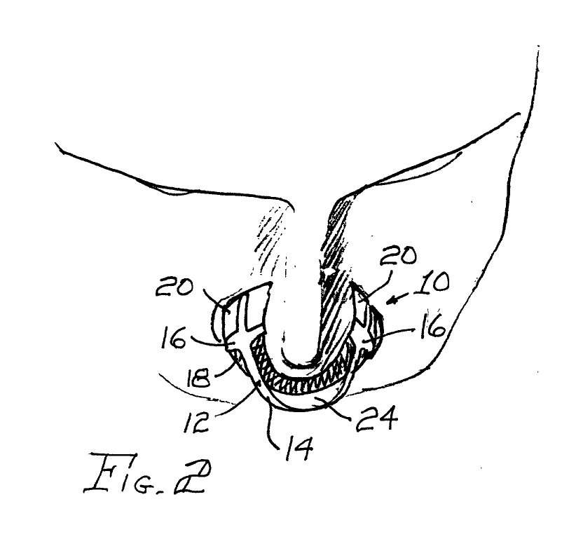

Spring 12 further includes larger end extension pads 20 affixed thereto at the extensions 16 and the terminal end of mid-strip 14 as by bonding or adhesive. Pads 20 are coated on their inner faces (toward the nose in use) with a medically approved adhesive of known type to cause the filter system to adhere to the nose on the outside of each nostril in use, as seen in FIGS. 1 and 2, and by means of the adhesive to exert outward pulling forces on the nostrils thereat in use. In usual manner, a release sheet 22 is provided over the adhesive on pads 20 to protect the adhesive until the filter system is used.

Further, the larger pads 20.provide sufficient protective coating surface area to facilitate manipulation of the filter system in placing the same in position on the nose.

By providing a relatively minimum size spring element 12, cost is obviously minimized, but more importantly, there is reduced likelihood of obstructing air flow to the nose with the filter in use as well as reduced weight, while still retaining the necessary structural strength.

The filter element, per se, comprises a length of filter material 18 of a length shorter than the spring material 12. Moreover, the filter material 18 is secured at only one end, as at the left in FIG. 4, to the spring material 12, as by adhesive, at the area of the spring material transverse extension 16, thereby to provide a larger surface area to secure and retain the filter material 18 on the spring material 12.

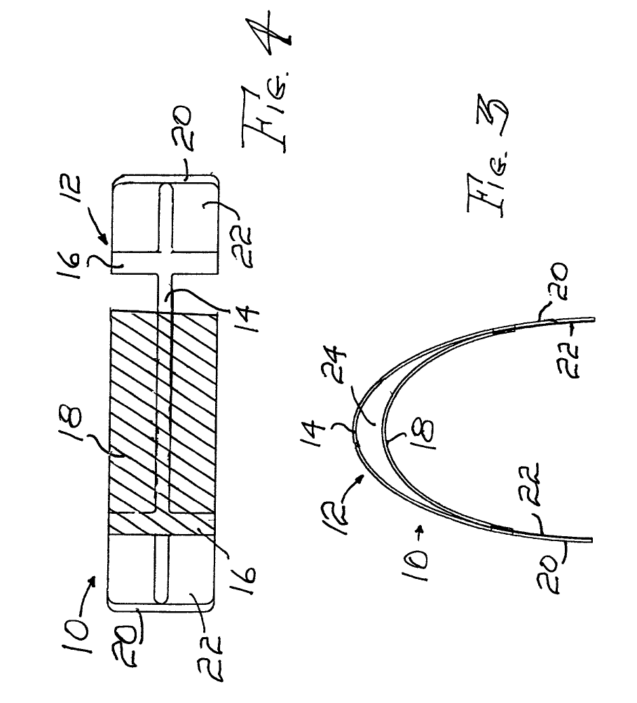

Additionally, and importantly, the length of the filter material 18 is less than the spacing between the spring extension portions 16. Accordingly, with the filter material 18 thus affixed to one spring extension 16, the spring material 12 in use is flexed to a generally open U-shape as seen in FIG. 3. In so doing, a small gap 24 will exist between the central portion of the filter material 18 and the outwardly adjacent central portion of the spring material 12 with the filter in position on the nose. Gap 24 also facilitates airflow to and through the filter material 18 and to the nose in use.

There are numerous and well-known filter materials suitable for the element 16 and which are capable of intercepting and filtering atmospheric dust, lint, dander, smoke, and diverse airborne pathogens to a quite small size, as on the order of 3 microns or less. Such filter material includes non-woven cottons, fine synthetic fibers, combinations thereof, and other available materials. One such material is that found on the Carrier 31MF Mechanical Air Cleaner, for example.

Accordingly, in use, and as seen in the drawings, the release sheets 22 are removed from the pads 20, and the plastic spine 14 is flexed to the position of FIG. 3, whereupon the filter system is affixed to the nose as seen in FIGS. 1 and 2, with the spine 14 extending under the nose with the filter material 18 between the spine 14 and the nose. By virtue of the free end of filter 18, the filter will accommodate to varying size noses and nostrils, without being stretched or binding against the nose. Rather, the filter is proximate the nostrils, and will especially engage the same as the user breathes in. In this manner, with the proximate positioning of the filter 18 with respect to the nose, effective removal of airborne dust and pathogens is effected before the same enter the nose.

While there is a small gap 24, as on the order of 0.2″, between the plastic spine 14 and the filter material 18, the filter 18 is normally in substantially direct contact with the nose at the nostrils. This assists in keeping the nose warm and moist in use, while the outward force F applied to the nostrils contributes to reduction in nasal congestion and favorably altering intranasal blood flow.

As a corollary thereto, the outward forces F exerted on the nostril by virtue of the spring spine 14 and the adherent nostril pads 20 will tend to keep the nasal passages fully open for enhanced breathing capability, as noted.

It will be seen from the foregoing that the improved nose filter system is exceedingly compact, lightweight, easily carried for ready use, and by virtue of low cost, is readily disposable. Further, the same in no way impedes normal speech or eating.

What I claim is:

1. A nose filter for airborne solids and pathogens comprising:
a length of flexible filter material,
a length of flexible spring material having a length greater than that of said filter material,
said filter material having one lateral end attached to said spring material at a point adjacent one end of the spring material, and,
an attachment system at the opposite ends of the spring material for attaching the nose filter to the nose on either side thereof externally of the nostrils,
whereby when said spring material is flexed to a generally U-form and the ends thereof attached to the nose, said filter material underlies the nostrils in slightly spaced relation to the spring material to facilitate breathing through said filter material to remove airborne solids and pathogens, and wherein said spring material exerts an outward force on the nose to insure that the nostrils are open.

2. The nose filter of claim 1 wherein the attachment system comprises adhesive on the ends of the spring material to facilitate attachment to the nose.

3. The nose filter of claim 2 wherein protective material covers the adhesive before use, and may be readily removed, thereby to attach the spring material to the nose externally of the nostrils.

4. The nose filter of claim 3 wherein the protective material are release sheets adhered to the adhesive.

5. The nose filter of claim 1 wherein the spring material has a width substantially less than that of the filter material.

6. The nose filter of claim 1 wherein the filter material blocks passage of airborne solids on the order of one to 3 microns in size.

7. The nose filter of claim 1 wherein the filter material includes non-woven cotton.

8. The nose filter of claim 1 wherein the filter material includes synthetic fibers.

9. The nose filter of claim 1 wherein the filter material includes non-woven cotton and synthetic fibers.

10. The nose filter of claim 1 wherein the spring material is plastic.

11. The nose filter of claim 10 wherein the plastic is a polyolefin.

12. The nose filter of claim 1 wherein the spring material is an elongated narrow strip having transversely extending portions adjacent each end thereof proving support for the attachment system.

13. The nose filter of claim 1 wherein the spring material is one the order of 2.20″ in length, 0.13″ along a narrower portion, and about 1.0″ wide at the opposite ends of the spring material.

14. A nose airflow enhancer and filter for airborne solids and pathogens comprising:
a length of spring material,
a length of filter material shorter than said spring material,
said filter material secured at one end to said spring material adjacent one end thereof,
and,
an attachment system at the ends of the spring material for attaching the same to the nose of a user on the outside of the nostrils with the filter material underlying the nostrils, thereby permitting the filter material to remove airborne solids and pathogens during breathing of the user while airflow through the nose is enhanced.

15. A nose airflow enhancer and filter for airborne solids and pathogens comprising:
a length of spring material, including a system for attaching the spring material when flexed to a U-shape to the outside of the nostrils, and,
a length of filter material carried by the spring material in position to underlie the nostrils when the spring material is attached to the nose, thereby permitting the filter material to remove airborne solids and pathogens during breathing of the user while airflow through the nose is enhanced.
